(19) United States
(12) Patent Application Publication
(10) Pub. No.: US 2024/0081768 A1
(43) Pub. Date: Mar. 14, 2024

(54) TECHNOLOGY FOR SAFETY CHECKING A MEDICAL DIAGNOSTIC REPORT

(71) Applicant: Siemens Healthcare GmbH, Erlangen (DE)

(72) Inventors: Sven KOHLE, Erlangen (DE); Volker SCHALLER, Uttenreuth (DE); Christoph SPEIER, Erlangen (DE); Christian TIETJEN, Fuerth (DE); Gerhard KOHL, Neunkirchen am Brand (DE); Bjoern NOLTE, Fuerth (DE)

(73) Assignee: Siemens Healthcare GmbH, Erlangen (DE)

(21) Appl. No.: 18/466,356

(22) Filed: Sep. 13, 2023

(30) Foreign Application Priority Data
Sep. 14, 2022 (EP) 22195490.2

(51) Int. Cl. A61B 6/00; G06N 20/00; G16H 30/40; G16H 50/70

(52) U.S. Cl. CPC A61B 6/5217; A61B 6/461; G06N 20/00; G16H 30/40; G16H 50/70

(57) ABSTRACT

A computer-implemented method comprises: reading in sensor data relating to an image file; assigning the sensor data to a first position of a patient's anatomical structure; extracting at least one second position of the patient's anatomical structure from a medical diagnostic report; comparing the first position and the at least one second position; and outputting a warning signal if the comparison does not yield a match.

### CROSS-REFERENCE TO RELATED APPLICATION(S)

The present application claims priority under 35 U.S.C. § 119 to European Patent Application No. 22195490.2, filed Sep. 14, 2022, the entire contents of which is incorporated herein by reference.

### FIELD

One or more example embodiments of the present invention relate to a computer-implemented method for safety checking a medical diagnostic report on the basis of an image file which was acquired using a medical imaging device. One or more example embodiments of the present invention further relate to a device, a system, a computer program product and/or a computer-readable storage medium which are each configured to carry out the method.

### BACKGROUND

In a radiological diagnostic investigation, peculiarities in image data from a current investigation are conventionally described in a diagnostic report and are often marked up or indeed measured in the current images. If the patient has undergone prior investigations using the same or indeed other investigation modalities, these are sometimes referred to for comparison.

It is important from a medico-legal standpoint for a diagnostic report to describe all the important disease processes which were identifiable in the images viewed. Information which is missing from the diagnostic report may result in diagnostic and/or treatment errors. It is for this reason that, for example in Germany, diagnostic reports are reviewed or supervised by senior physicians.

Despite this duplicate assessment of the images and diagnostic report, disease processes are overlooked. It is moreover in the first reporting physician's interest for the text of the diagnostic report already to be as error-free and complete as possible when it is reviewed.

A radiological image frequently reveals further secondary findings in addition to the main finding. In particular when a radiological image includes numerous secondary findings, it is entirely possible for the reporting radiologist to forget to mention one or more secondary findings in the text of the diagnostic report.

At present, the diagnostic report is only manually reviewed by a second radiologist. However, the susceptibility to error nevertheless subsists, in particular where there are a number of secondary findings.

### SUMMARY

It is an object of one or more example embodiments of the present invention to provide technology which ensures that a diagnostic report contains all relevant information, in particular with regard to all the viewed image areas, and/or both the main finding and any secondary findings. It is alternatively or additionally an object of one or more example embodiments of the present invention to improve the interpretation of an image file and thus enable an improvement in the quality of diagnosis and/or a device-based treatment and/or treatment strategy.

At least this object may be achieved in each case by subject matter according to the equal-ranking independent claims. Advantageous embodiments may constitute the subject matter of the dependent claims, the description and the drawings.

According to one aspect of the method, a computer-implemented method is provided for safety checking a medical diagnostic report for a patient on the basis of an image file, wherein the image file was acquired using a medical imaging device. The method comprises the step of reading in sensor data relating to the image file. The method further comprises the step of assigning the read-in sensor data to a first position of a patient's anatomical structure. The method further comprises the step of extracting at least one second position of a patient's anatomical structure from the medical diagnostic report. The method further comprises the step of comparing the first position and the at least one second position. The method further comprises the step of outputting a warning signal if the comparison does not yield any matches.

The sensor data relating to the image file may define a focus area. The focus area may be the focus of viewing the image file, in particular of viewing the first position, by trained medical personnel, in particular a physician. For example, the sensor data may provide one or more indications of viewing, in particular of the first position, by trained medical personnel, in particular a physician. Alternatively or additionally, the trained medical personnel, in particular the physician, may be the author of the medical diagnostic report.

The indication or indications of viewing may comprise, in particular digitally, one or more movements and/or inputs carried out on the image file by way of a user interface. The movements and/or inputs may for example comprise computer mouse movements, trackpad movements, trackball movements, key movements (e.g. by way of keyboard keys), swiping movements on a touch-sensitive screen and/or inputs by way of the corresponding input device and/or means (e.g. computer mouse, trackpad, trackball, joystick, keyboard, and/or touch-sensitive screen). The movements and/or inputs may, for example, comprise zooming in on an area which may also be denoted focus area.

Alternatively or additionally, the inputs may for example comprise measurements (e.g. distance measurements and/or size measurements of an organ) on the image file and/or annotations (e.g. mark-ups by way of simple graphical symbols and/or short texts) on the image file.

Alternatively or additionally, the indication or indications of viewing may be determined by way of eye tracking.

The image file may furthermore alternatively or additionally comprise an image stack of slices. A heatmap may comprise a duration of the viewing of individual ones or all of the slices of the image stack. For example, a slice viewed for a long time (and thus intensively) or an area in the image subjected to long/intense analysis may be assigned to a focus area.

Reading in sensor data relating to the image file may be suitable for determining the focus area. Alternatively or additionally, reading in the sensor data relating to the image file may include determining the focus area.

The focus area may correspond to an area of the image file which was the focus of viewing by the trained medical personnel, in particular the physician, and/or for which there are one or more indications of viewing by the trained medical personnel, in particular the physician.

The focus area may comprise the first position of the patient's anatomical structure and/or be assigned thereto. The first position of the patient's anatomical structure may comprise or identify a healthy structure, a suspect area, and/or a lesion (also: abnormal structure or diseased structure). The focus area may for example correspond to an area of the image file which shows a possibly anomalous tissue structure or lesion of the patient. A tissue structure may possibly be classified as a lesion due to a color and/or transparency, an opacity and/or a shape in an area of the image file, in particular (at) the first position.

A text and/or coding scheme may be assigned to the first position.

The diagnostic report may also be denoted physician's report. The diagnostic report may comprise an assessment of the image file by the trained medical personnel, in particular the physician, with regard to classification as healthy structures, suspect areas, and/or lesions.

The diagnostic report may comprise running text and/or a coding scheme. The coding scheme may comprise the at least one second position. Alternatively or additionally, the coding scheme may comprise a description and/or a classification of the at least one second position.

The at least one second position may be extracted from the running text and/or the coding scheme.

The comparison of the first position and the at least one second position may yield a match. For example, the trained medical personnel, in particular the physician, may have viewed, measured and/or annotated a first position as the focus area of the image file. The trained medical personnel, in particular the physician may have mentioned this focus area in the diagnostic report. Mentioning the focus area in the diagnostic report may be denoted mentioning the second position.

The read-in sensor data may result in the assignment of a plurality of first positions to the patient's anatomical structure. The computer-implemented method and/or the comparison with the at least one second position of the patient's anatomical structure may be carried out for each of the plurality of first positions.

Alternatively or additionally, the first position and the at least one second position may not match (e.g. not sufficiently). If the comparison yields a non-match (e.g. insufficient match), a warning signal may be output. A match is in particular not necessarily taken to mean identity of the positions. The match may, for example, be ascertained on the basis of a match score, wherein the match score may describe an absolute or relative distance between the first and the at least one second position, for example a distance in centimeters or pixels. A match is for example taken to mean a match score and/or distance of less than a threshold value. The threshold value may be an absolute threshold value, a position-dependent threshold value or an organ-specific threshold value.

The warning signal may comprise a visual indication on a screen, for example an error message on quitting an editing program (also: data processing program) for the diagnostic report, on attempting to initial the diagnostic report, and/or on the basis of some other event.

Alternatively or additionally, the checking method may be initiated by the author of the diagnostic report and/or by a checker (e.g. in the course of quality control by a second physician and/or by a quality control unit of a medical facility to which the medical imaging device and/or the image file is assigned), for example by way of a button on an input screen.

Alternatively or additionally, the quality control method may be automatically started, for example at an arbitrary and/or predetermined point in time after the author has signed the diagnostic report.

Furthermore the warning signal may alternatively or additionally be output continuously, in particular during drafting of the diagnostic report, until the (or each) first position matches with a (for example respective) corresponding second position in the diagnostic report.

The warning signal and/or visual indication on the screen may comprise an error message, in particular in text format. Alternatively or additionally, the indication on the screen may comprise a reproduction of the image file marked up (e.g. in red) for the (or each) first position, the comparison of which with the second positions has not yielded a match in the diagnostic report.

Alternatively or additionally, the image file may be continuously reproduced on the screen marked up (e.g. in green) for the (or each) first position, for which comparison with the second positions has yielded a match in the diagnostic report.

In particular, matches can be marked up on a reproduction in a first coding scheme, for example a color, and/or by way of a first visual indication. Missing matches can be marked up on the reproduction in a second coding scheme, in particular color, and/or by way of a second visual indication. The first coding scheme, in particular color, and the second coding scheme, in particular color, may be different, for example green or red. Alternatively or additionally, the first visual indication and the second visual indication may be different (e.g. with regard to shape), for example comprise outlines with different kinds of lines (e.g. continuous vs. dashed, or vice versa).

Alternatively or additionally, the warning signal may comprise an acoustic signal which is output for example in the event of predetermined actions by the trained medical personnel, in particular the physician. For example, an acoustic warning signal can be output on (e.g. interim) closure of the editing program with which the diagnostic report is being drafted and/or edited, and/or if an attempt is made to sign the diagnostic report.

By way of the method, in particular by comparing the sensor data and mentioning positions in the diagnostic report, it is possible to ensure that the diagnostic report is comprehensive and/or mentions all potentially relevant positions. Alternatively or additionally, the method can improve diagnostic quality thanks to or on the basis of the image file. An improved diagnosis in turn enables an improved treatment or therapeutic strategy.

The sensor data may comprise a duration of display, in particular on a screen, for example of an image slice within an image stack of medical images of the image file. Alternatively or additionally, the sensor data may comprise movement data from an input device and/or means, in particular a computer mouse, on a display of an image or image slice of the image file. Furthermore alternatively or additionally, the sensor data may comprise movement data from gaze detection on a display of an image or image slice of the image file. Furthermore alternatively or additionally, the sensor data may comprise mark-ups of a region of interest on a display of an image or image slice of the image file. Furthermore alternatively or additionally, the sensor data may comprise measurements of a distance, a diameter and/or a density on a display of an image or image slice of the image file. Furthermore alternatively or additionally, the sensor data may comprise annotations about a display of an image or image slice of the image file. Furthermore alternatively or additionally, the sensor data may comprise operating data regarding a display of at least one image of the image file.

The movement data may also be denoted movement patterns and/or operating patterns.

A collection (and/or arrangement) of display durations (e.g. on a screen for viewing by the trained medical personnel, in particular the physician) may be denoted and/or displayed as a heatmap of the image slices of the image stack.

Gaze detection may also be denoted “eye tracking”.

The operating data of the display of the at least one image of the image file may comprise zooming in on a subarea of the image, in particular comprising the first position.

Assigning the read-in sensor data to the first position of the patient's anatomical structure may comprise identifying anatomical landmarks, or segmenting, in particular of at least one of the patient's organs. Alternatively or additionally, the assignment may be made by way of an algorithm trained by machine learning.

The anatomical landmark may comprise a conspicuous and/or prominent anatomical structure, for example the patient's spinal column and/or parts of their skeleton. Segmentation may comprise segmenting an organ or an anatomical subarea, for example of the lung, liver or a kidney.

The algorithm trained using machine learning may comprise artificial intelligence (AI). Alternatively or additionally, the machine learning may comprise one or more neural networks, in particular a convolutional neural network (CNN).

The algorithm may be trained using annotated image files, in particular obtained from the medical imaging device or a device of the same kind. Alternatively or additionally, the trained algorithm may identify the first position without intermediate steps, in particular identification of anatomical landmarks, and/or without segmentation.

Assigning the read-in sensor data to the first position of the patient's anatomical structure may comprise a step of outputting the result of assigning in a coding scheme. The coding scheme may optionally comprise SNOMED and/or RadLex.

SNOMED may be taken to be a computer-processable ontology or collection of medical terms, codes and thus to be a standard for health terminology or systematized nomenclature (also: healthcare terminology). SNOMED may comprise different subdivisions, in particular SNOMED-CT (clinical terms), inter alia also for medical imaging methods.

RadLex may be taken to be a standardized or controlled terminology for radiology. RadLex may harmonize and/or supplement other lexicons and/or standards, for example SNOMED-CT, LOINC and/or DICOM. In addition to radiological terms, RadLex may define dependencies and interrelationships of the terms.

Alternatively or additionally, the coding scheme may comprise LOINC and/or DICOM.

Extracting the at least one second position of the patient's anatomical structure from the medical diagnostic report may comprise linguistic data processing of a text of the medical diagnostic report and subsequent application of a coding scheme to the at least one second position extracted from the linguistically data-processed text. The coding scheme may optionally comprise SNOMED and/or RadLex.

Extracting the at least one second position of the patient's anatomical structure from the medical diagnostic report may comprise output in a coding scheme. The coding scheme may optionally comprise SNOMED and/or RadLex.

Comparing the first position assigned to the read-in sensor data with the at least one second position extracted from the medical diagnostic report may comprise a computer-based or algorithmically performed comparison of the respective coding schemes. The respective coding scheme may optionally comprise SNOMED and/or RadLex.

The warning signal may comprise output, on a display of an image or a slice of a 3D image file, of the first position assigned to the read-in sensor data for which the step of comparing did not identify a corresponding second position extracted from the medical diagnostic report. Alternatively or additionally, the warning signal may comprise text output of a missing match with regard to the first position assigned to the read-in sensor data. Furthermore alternatively or additionally, the warning signal may comprise blocking a working step of completing and/or signing the medical diagnostic report.

Blocking the working step of completing and/or signing the medical diagnostic report may depend on an assessment of the importance of the missing match. For example, the importance may be high if the sensor data indicate that the first position was intensively viewed, measured and/or annotated by the trained medical personnel, in particular the physician.

The importance of the missing match may be low if the sensor data indicate that the first position was viewed for only a very short period or was not measured and/or not annotated.

Depending on the exemplary embodiment, the warning signal may be output at different points in time during drafting of the diagnostic report.

For example, the warning signal may be output continuously, and/or on the basis of a check query to the author of the diagnostic report, during drafting of the diagnostic report.

Alternatively or additionally, the warning signal may be output on (e.g. interim) closure of the editing program of the diagnostic report and/or if an attempt is made to complete and/or sign the diagnostic report.

Alternatively or cumulatively, the warning signal may be generated during quality control of the already completed and/or signed diagnostic report.

Quality control may, for example, be performed outside the reading and reporting working procedure of the trained medical personnel, in particular the physician, centrally on a machine, for example a server, of a hospital information system (HIS) and/or a radiology information system (RIS).

Output of the warning signal may comprise a request to correct and/or amend the medical diagnostic report. The quality of the diagnostic investigation may thus be instantaneously improved.

The medical imaging device may comprise an X-ray machine. Alternatively or additionally, the medical imaging device may comprise a magnetic resonance tomograph (MRT). Furthermore alternatively or additionally, the medical imaging device may comprise a positron emission tomograph (PET). Furthermore alternatively or additionally, the medical imaging device may comprise an angiography system (AX). Furthermore alternatively or additionally, the medical imaging device may comprise a computed tomograph (CT). Furthermore alternatively or additionally, the medical imaging device may comprise an ultrasound scanner.

The method may further comprise a step of reading in further patient data, wherein the further patient data has been collected independently of the image file.

The method may further comprise a step of extracting at least one third position of a patient's anatomical structure from the read-in further patient data.

The method may further comprise a step of comparing the at least one third position extracted from the further patient data with the first position and/or with the at least one second position.

The method may further comprise a step of outputting an indication, in particular in the form of an indication signal, if the comparison yields a match, wherein the indication is directed toward reviewing the consistency of the diagnostic report with the read-in further patient data.

By reading in the further patient data, extracting one or more positions from the read-in further patient data and making a comparison with the first position from the sensor data and/or with the second positions from the diagnostic report, it is possible to consult the patient's prior medical history in order to arrive at a diagnosis. As a result, the quality of the medical diagnosis and/or the diagnostic report can be further improved.

The indication can draw the attention of trained medical personnel, in particular a physician, to the fact that there is prior medical history which relates to the first position of the anatomical structure.

The method may further comprise a step of reading in a medical guideline (which may also be denoted medical directive and/or standard of operation (SOP)) which is classed as at least potentially relevant to the image file, the read-in sensor data and/or the first position of the patient's anatomical structure.

The method may further comprise a step of reviewing the medical diagnostic report with regard to compliance with the medical guideline.

The method may further comprise a step of outputting a warning signal if the medical diagnostic report is not, or not fully, compliant with the guideline. It is also possible for the degree of deviation to be encoded and stored and made retrievable.

The medical guideline may also be denoted “standards of operation (SOP)”.

The medical diagnostic report may comprise a sequence of diagnostic steps which may have taken place before and/or may yet take place after capture of the image file. Reviewing the medical diagnostic report with regard to compliance with the medical guideline may comprise a review as to whether, on the basis of the medical diagnostic report, diagnostic steps according to the medical guideline have been or are still being observed. Alternatively or additionally, reviewing the medical diagnostic report with regard to compliance with the medical guideline may comprise a review as to whether predetermined combinations of specialist medical terminology are present in the medical diagnostic report. The specialist medical terminology may relate to one or more positions of an anatomical structure, in particular of the first position, a diagnosis and/or a therapeutic option.

The warning signal can be output in the event of a deviation from the medical guideline, for example in the absence of a further indicated differential diagnosis and/or treatment plan being provided.

The quality of diagnosis and treatment planning can be further improved by the review of the medical diagnostic report with regard to compliance with the medical guideline.

The step of reviewing the medical diagnostic report with regard to compliance with the medical guideline may further comprise identifying at least one anatomical structure in the medical guideline and comparing the identified anatomical structure with the anatomical structure of the at least one first position and/or comparing the identified anatomical structure with the anatomical structure of the at least one second position.

The step of reviewing the medical diagnostic report with regard to compliance with the medical guideline may further comprise identifying at least one fourth position of an anatomical structure in the medical guideline and comparing the at least one fourth position with the at least one first position and/or comparing the at least one fourth position with the at least one second position.

The above-stated steps enable automatic identification in the guideline of those anatomical structures which are to be investigated in the diagnostic investigation of the image file according to the guideline. Reconciliation with the first or second position information or the associated anatomical structures enables an automatic review of whether a), on the basis of the sensor data, the anatomical structures have even been looked at and/or b) whether the anatomical structures have been addressed the report.

The step of reviewing the medical diagnostic report with regard to compliance with the medical guideline may further comprise outputting a warning message on the basis of the step of comparing the identified anatomical structure with the anatomical structure of the at least one second position or of comparing the at least one fourth position with the at least one first position and/or the at least one second position.

As a result, a warning message can automatically be output if, contrary to the medical guideline, an anatomical structure has not been included in the diagnostic report.

Identifying the patient's at least one anatomical structure of the at least one fourth position from the medical guideline may comprise linguistic data processing of a text of the medical guideline and subsequent application of a coding scheme to the at least one anatomical structure extracted from the linguistically data-processed text. The coding scheme may optionally comprise SNOMED and/or RadLex.

The step of reviewing the medical diagnostic report with regard to compliance with the medical guideline may comprise an algorithm trained by machine learning and/or artificial intelligence (AI).

Comparison with the medical guideline may be carried out in an automated manner and/or on a central machine, for example the KIS and/or RIS server.

The preceding description relates to the manner in which the object was achieved with regard to the method. Features, advantages or alternative embodiments mentioned in this connection are likewise also to be applied to the other claimed subjects (in particular the device, system, computer program product and/or computer-readable storage medium) and vice versa. In other words, the substantive claims (e.g. directed to a device, a system or a computer program product) can also be further developed with the features which are described or claimed in connection with the method and vice versa. The corresponding functional features of the method are here embodied by corresponding substantive modules (e.g. in each case denoted “unit” or “interface”), in particular by hardware modules or microprocessor modules, of the device, system or product and vice versa.

One aspect of the device provides a device for safety checking a medical diagnostic report for a patient on the basis of an image file, wherein the image file was acquired using a medical imaging device. The device comprises a sensor interface which is configured to receive sensor data relating to the image file from at least one sensor. The device further comprises an assignment unit which is configured to assign the read-in sensor data to a first position of a patient's anatomical structure. The device further comprises an extraction unit which is configured to extract at least one second position of a patient's anatomical structure from the medical diagnostic report. The device further comprises a comparison unit which is configured to compare the first position and the at least one second position. The device further comprises an output interface which is configured to output a warning signal if the comparison does not yield a match. According to one aspect, the device is configured to carry out the above-described method or one of the variants thereof.

The at least one sensor may comprise a motion sensor, in particular an input interface, and/or an input sensor of an input interface, wherein the input interface may comprise a computer mouse, a trackpad, a trackball, a joystick, a keyboard, and/or a touch-sensitive screen. Alternatively or additionally, the at least one sensor may comprise an eye tracking sensor. Alternatively or additionally, the at least one sensor may comprise a timer which is configured to measure the duration of viewing of a slice from an image stack or an area in the image which is encoded in the image file.

The device may further be configured to carry out the steps of the method according to the method aspect, and/or features of the method according to the method aspect.

A further aspect provides a system for safety checking a medical diagnostic report. The system comprises at least one sensor for acquiring sensor data relating to an image file. The system further comprises a device according to the device aspect, wherein the sensor interface of the device reads in the sensor data from the at least one sensor. The system further optionally comprises a warning signal output mechanism, device and/or means, which outputs the warning signal on the basis of the warning signal provided at the output interface of the device.

A further aspect provides a computer program product comprising commands which, on execution of the program by a computer, cause the latter to carry out the method according to the method aspect.

A further aspect provides a computer-readable storage medium comprising commands which, on execution by a computer, cause the latter to carry out the method according to the method aspect.

### BRIEF DESCRIPTION OF THE DRAWINGS

The following detailed description of the figures discusses, with reference to the drawings, exemplary embodiments, which are not intended to be understood in limiting manner, with the features and further advantages thereof. In the drawings:

FIG. 1 shows a flowchart of a method according to a preferred embodiment of the present invention;

FIG. 2 shows a block diagram of a device according to a preferred embodiment of the present invention; and

FIG. 3 shows an exemplary clinical working procedure during which the steps of the method according to embodiments of the present invention can be carried out.

### DETAILED DESCRIPTION

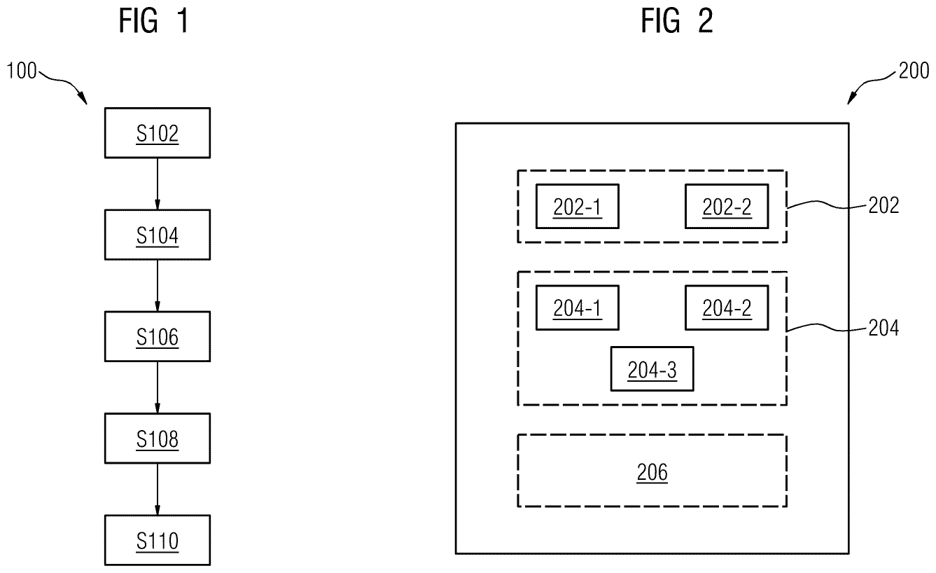

FIG. 1 shows an exemplary computer-implemented method for safety checking a medical diagnostic report for a patient on the basis of an image file, wherein the image file was acquired using a medical imaging device. The method is denoted overall with reference sign 100.

In a step S102, sensor data relating to the image file is read in. In a step S104, the read-in sensor data is assigned to a first position of a patient's anatomical structure. In a step S106, at least one second position of a patient's anatomical structure is extracted from the medical diagnostic report. In a step S108, the first position is compared with the at least one second position. Finally, in a step S110, a warning signal is output if the comparison S108 does not yield or has not yielded a match.

FIG. 2 shows an exemplary device for safety checking a medical diagnostic report for a patient on the basis of an image file, wherein the image file was acquired using a medical imaging device. The device is denoted overall with reference sign 200.

The device 200 shown in FIG. 2 comprises a sensor interface 202-1 which is configured to receive sensor data relating to the image file from at least one sensor. The device 200 shown in FIG. 2 further comprises an assignment unit 204-1 which is configured to assign the read-in sensor data to a first position of a patient's anatomical structure. The device 200 shown in FIG. 2 further comprises an extraction unit 204-2 which is configured to extract at least one second position of a patient's anatomical structure from the medical diagnostic report. The device 200 shown in FIG. 2 further comprises a comparison unit 204-3 which is configured to compare the first position and the at least one second position. The device 200 shown in FIG. 2 further comprises an output interface 202-2 which is configured to output a warning signal if the comparison does not yield a match.

According to one exemplary embodiment, which is combinable with any other exemplary embodiment, the sensor interface 202-1 and the output interface 202-2 can be combined in a data interface 202 of the device 200.

According to a further exemplary embodiment, which is combinable with any other exemplary embodiment, the assignment unit 204-1, the extraction unit 204-2 and the comparison unit 204-3 can be arranged in an (in particular common) computing unit and/or a processor 204.

According to a further exemplary embodiment, which is combinable with any other exemplary embodiment, the device 200 may further comprise a data storage device and/or means 206.

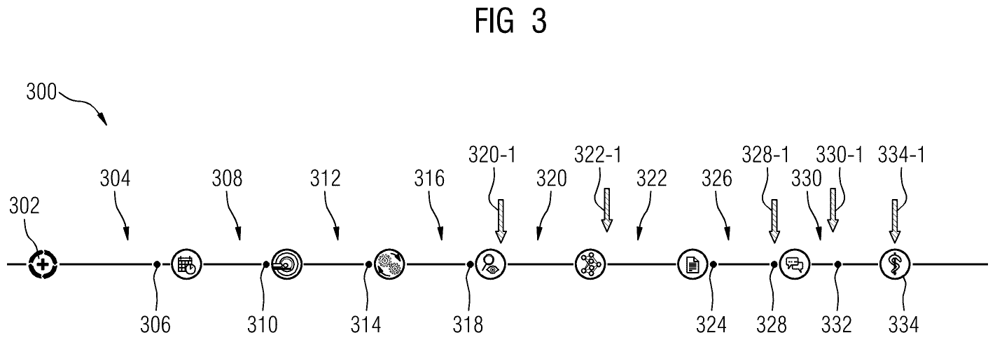

FIG. 3 shows an example of a workflow 300 during which the method according to embodiments of the present invention is carried out.

During the conventional diagnostic investigation of radiological image files (for short: images), the trained medical personnel (also: physician and/or user of the image viewing software), making use of image viewing software, focuses on conspicuous image areas which are indicative of a disease process and/or which demand particular attention due to a medical problem.

According to an embodiment of the present invention, the following sensor data (which may also be denoted information) may be used to understand which image areas the trained medical personnel (also: physician and/or user of the image viewing software) has focused on while reading the diagnostic report. Image areas on which the trained medical personnel had focused while viewing the image file and/or reading the diagnostic report may also be denoted focus areas.

According to a first exemplary embodiment, which is combinable with any other exemplary embodiment, a heatmap is generated (e.g. as sensor data) from the longer-viewed slices in an image stack. One or more focus areas can be determined from the heatmap.

Alternatively or additionally, movement data from an input device and/or means, and/or gaze detection (e.g. also: mouse tracking and/or eye tracking) may be used (e.g. as sensor data) for identifying focus areas (also: focused-on image areas) on a slice (also: cross-sectional image plane) or on a two-dimensional image.

According to a second exemplary embodiment, which is combinable with any other exemplary embodiment, manual measurements and/or annotations, in particular a “region of interest” (ROI) or distance measurement, are used (e.g. as sensor data) for determining a focus area. Alternatively or additionally, values obtained from the measurements, for example a diameter and/or density (e.g. of an anatomical structure, in particular of an organ or lesion), may be used for determining the focus area.

According to a third exemplary embodiment, which is combinable with any other exemplary embodiment, one or more focus areas can be determined (e.g. in each case as sensor data) by running a specific utility (also: tool) and/or using another operating pattern. The specific utility may comprise a “medical imaging interaction toolkit” (MITK) software utility and/or for example segmentation and/or annotations.

In order to assign these data points to an (e.g. first) position of an anatomical structure (for short: anatomical position; also: anatomical location), an algorithm can assign the areas and/or points relating to the image file (or the image) to the (e.g. first) anatomical positions (and/or anatomical locations). This may proceed, for example, by way of (e.g. ALPHA) landmarks, (in particular organ) segmentation and/or a machine learning (ML) algorithm explicitly trained (e.g. with deep learning (DL)) for this purpose. The (e.g. first) position of the anatomical structure (and/or anatomical location) is here advantageously output in a common coding scheme format (e.g. SNOMED and/or RadLex).

If the focused-on image areas (and/or sensor data) are acquired during the diagnostic investigation and assigned to one or more (e.g. first) positions of anatomical structures (and/or anatomical locations), it is possible to review whether the diagnostic report contains a match (also denoted semantic correlate) for each focused image area (and/or for all the sensor data).

A match (and/or a semantic correlate) may be reviewed depending on the nature of the diagnostic report. A distinction is drawn below between largely unstructured diagnostic reports and structured diagnostic reports.

For unstructured diagnostic reports (in particular largely running text, for example dictated), natural language processing is used in a first step to identify which (e.g. at least one second) positions of anatomical structures (and/or which anatomical locations) are already mentioned in the diagnostic report.

In a second step, the (e.g. at least one second) positions of anatomical structures (and/or extracted anatomical location) extracted from the diagnostic report are translated into an, in particular common, coding scheme (e.g. SNOMED and/or RadLex).

In a third step, the encoded (e.g. at least one second) positions of anatomical structures (and/or anatomical locations) from the text of the diagnostic report are compared with the one or more (e.g. first) positions of anatomical structures (and/or anatomical locations) from the sensor data (and/or from the focused-on image areas).

For structured diagnostic reports (in particular comprising ordered text and structured encoded metainformation), the (e.g. at least one second) positions of anatomical structures (and/or anatomical locations) are already present in encoded form (e.g. according to a standard) in the structured diagnostic report.

In one step, the encoded (e.g. at least one second) positions of anatomical structures (and/or encoded anatomical locations) from the structured diagnostic report are compared with the one or more (e.g. first) positions of anatomical structures (and/or anatomical locations) from the sensor data (and/or from the focused-on image areas).

If matches and/or correlates to focused-on image areas and/or to one or more (e.g. first) positions of anatomical structures, to which sensor data is assigned, are missing from the current diagnostic report, a user, in particular trained medical personnel and/or a physician can be informed of this in various ways.

According to one exemplary embodiment, which is combinable with any other exemplary embodiment, when an attempt is made to complete the diagnostic report (and/or on signing), all those first positions of anatomical structures (and/or focused-on image areas) for which there is no match and/or no semantic correlate in the diagnostic report are listed and visualized. Only once the author of the diagnostic report (and/or the signatory and/or user) has confirmed that the omission of the match (and/or semantic correlate) was intentional is the diagnostic report finalized.

According to a further exemplary embodiment, which is combinable with any other exemplary embodiment, during diagnostic reporting a schematic view of the viewed body area is displayed to the author (and/or user), in which first positions of anatomical structures (and/or focused-on image areas) which are not mentioned in the diagnostic report are marked up with a signal color (e.g. red). First positions of anatomical structures (and/or focused-on image areas) which have already been mentioned in the diagnostic report are either not marked up or are marked up with a success color (e.g. green). The success color advantageously differs from the signal color.

According to a further exemplary embodiment, which is combinable with any other exemplary embodiment, during diagnostic reporting a warning symbol is displayed to the author (and/or user) in the diagnostic report, for example at the side, which indicates (and/or outputs an appropriate warning signal) when a computer mouse is passed (and/or hovered) over first positions of anatomical structures (and/or focused-on image areas) which have no match and/or no semantic correlate in the text of the diagnostic report. The author (and/or user) can jump to the first position and/or focused-on image area with a single click. In a structured diagnostic report, these warning signals (also: warning symbols) can be placed in targeted manner at those points in the diagnostic report and/or in the diagnostic report template where a corresponding semantic correlate would be expected.

Alternatively or additionally, the technology (in particular the method carried out in the system) can also warn if an artificial intelligence result (also: AI result) has been neither accepted nor rejected by the author (and/or user).

The technology according to embodiments of the present invention (in particular the described method, the device and/or the system) assists reporting trained medical personnel, in particular radiologists, in drafting a complete and consistent diagnostic report. The technology according to embodiments of the present invention introduces a further (e.g. in addition to conventional) safety level which makes it possible to improve the quality of diagnostic reports and minimize diagnostic investigation errors.

Increasing the quality of diagnostic reports and minimizing diagnostic investigation errors has patient benefits since any possible treatment errors arising from a misdiagnosis can be minimized. The technology according to embodiments of the present invention moreover accelerates the review (and/or supervision) process since the supervising senior physician more rarely has to make subsequent corrections.

Medical guidelines and/or standards of operation (SOP) contain recommendations and/or rules about how specific patient medical data should be analyzed in order to make or exclude a specific diagnosis. Medical guidelines and/or SOPs can state which diagnostic steps and/or measurements should be carried out, and/or which data processing tools should be applied to the available data (in particular image file data and/or the sensor data) when a specific medical condition of a patient is being diagnosed. Moreover medical guidelines may establish what follow-up action should be taken depending on the results of the diagnostic steps. Some medical guidelines contain information about the prevention and the prognosis of specific diseases and about risks and/or benefits.

Guidelines developed by governments, medical and professional groups and other bodies are intended to avoid unnecessary tests and investigations as well as overtreatment and/or undertreatment. Failure to observe guidelines may lead to suboptimal and/or harmful diagnostic and/or therapeutic follow-up action.

The information in a guideline is generally specific to a particular medical discipline and/or to a specific health condition and/or the available patient medical data. As a consequence, clinical guidelines are usually highly complex. For example, the current treatment flowchart published by the US National Comprehensive Cancer Network (NCCN) runs to almost 200 pages. It is therefore usually difficult for doctors to comply with these guidelines when reading a patient's case because it is no easy task to memorize and apply the guidelines in their entirety. In particular, when a radiologist working at a reading and diagnostic station (e.g. a computer and/or a device 200) is faced with a non-standard case, it is conventionally very difficult to apply the guidelines correctly. Moreover, a user (e.g. trained medical personnel, in particular a radiologist) may be tempted as a matter of clinical routine normally to pass over certain test points of a guideline in order to save time, being unaware of what the omission of a specific step might mean for the further clinical pathway.

This problem is conventionally solved by drawing the user's attention to the corresponding guideline before or while a patient's case is read. One problem with this conventional approach is that a user often does not have the time in their daily routine to familiarize themselves with the guidelines in order to establish which steps need to be applied. Furthermore, expert users in particular may feel patronized by such measures as they generally (e.g. in standard cases) know what they have to do to comply with guidelines or where slight deviations are possible without causing problems for downstream diagnosis or treatment decisions for the patient.

According to an exemplary embodiment which is combinable with any other exemplary embodiment, the technology proposed here automatically identifies whether and in what way a user (e.g. trained medical personnel, in particular a radiologist) is deviating from the applicable medical guideline when reading a patient's case and in particular medical image data (e.g. comprising the image file and/or the sensor data). For this purpose, the actions performed by a user (e.g. trained medical personnel, in particular a radiologist) and the conclusions drawn by him/her can be automatically analyzed for their match with the applicable medical guideline.

The method may comprise a step of accessing the patient data model and reading out available patient data (e.g. image data, in particular comprising the image file and the associated sensor data, non-image data, earlier reports, a worklist entry for the user, and/or task for the user).

The method may further comprise a step of identifying the applicable medical guideline and/or SOP on the basis of the available patient data.

The method may further optionally comprise a step of determining the patient's stage (e.g. with regard to a medical condition and/or a treatment) in the identified guideline.

The method may further comprise a step of monitoring user actions by directly monitoring user inputs in the reading and diagnostic station (e.g. the computer and/or the device 200) and/or by analyzing the user's medical report once it has been initialed (also: signed).

It may be reviewed which utilities (also: tools) were used. Alternatively or additionally, it may be reviewed whether artificial intelligence (AI)-based results from AI-based utilities (also: AI-based tools) are available. If AI-based results are available, at least one confidence value (also: confidence level) may be reviewed. Furthermore, it may alternatively or additionally be reviewed which elements of the available patient data were opened and/or consulted.

Furthermore, it may alternatively or additionally be reviewed which measurements were made. Furthermore, it may alternatively or additionally be reviewed which measurements were taken into account in the report. Furthermore, it may alternatively or additionally be reviewed what diagnosis was stated. Furthermore, it may alternatively or additionally be reviewed which differential diagnosis or differential diagnoses were taken into account. Furthermore, it may alternatively or additionally be reviewed which treatment options are recommended. Furthermore, it may alternatively or additionally be reviewed, which follow-up investigations were taken into account or are to be taken into account in future.

The method may further comprise a step of comparing the monitored user action (e.g. the diagnosis and/or treatment steps) with the ascertained guideline while taking account of the ascertained stage.

The method may further comprise a step of ascertaining a deviation from the guideline. A deviation may involve an (e.g. AI) utility (also: tool) not being applied to the patient data.

Alternatively or additionally, a deviation may involve a measurement not being made.

Alternatively or additionally, a deviation may involve some aspects of the patient data not being taken into account, for example non-image data such as laboratory data, specific portions of an image study and/or an available prior study.

Alternatively or additionally, a deviation may mean that no meaningful assessments (also: scores), for example breast imaging-reporting and data system (BI-RADS) features, can be extracted from a report.

Alternatively or additionally, a deviation may involve additional tests not provided in the SOP being carried out and/or planned. Alternatively or additionally, a deviation may involve non-optimal tests (e.g. MRT instead of ultrasound as imaging mode) being carried out and/or planned.

The method may further optionally comprise a step of determining a criticality of a deviation from the guideline. The criticality of the deviation is here preferably quantified and/or converted into an outputtable format. Some omissions and/or deviations from the guideline, for example of secondary diagnoses, possibly have only a limited impact on patient flow (also: patient journey or patient pathway), while other omissions and/or deviations from the guideline may put the patient on a completely different (in particular therapeutic treatment) pathway. Criticality may for example be estimated by a number of nodes which, in the event of incorrect use of the guidelines, would proceed differently than in a probable course.

Depending on criticality, the method may further comprise a step of informing the user that a deviation from the guideline (in particular with a specific criticality) is present, and/or which anatomical areas or what must be reviewed in addition to the user actions, in order to reverse the deviation from the guideline. Depending on criticality (e.g. at a criticality above a predetermined threshold value), a medical report previously initialed by the user may be rejected until the deviation has been remedied.

The method may further comprise a step of determining the consequences of the deviation from the guideline for further patient flow and optionally draw the user's attention to the consequences in order to encourage the user to understand that specific critical deviations from the guideline must be remedied by way of an explicable (in particular AI) utility (also: tool). Such consequences might for example comprise the need for a renewed investigation of the patient and/or a treatment recommendation based on diagnostic reports with a lower confidence value (also: lower confidence level).

The method may further involve, in the event of the deviation being considered reasonable, for example due to information not acquired in the electronic patient record, the necessary documentation with regard to added or omitted steps together with reasons stated by the trained medical personnel, in particular a physician in charge, being automatically performed in the patient record.

FIG. 3 shows exemplary stages of a clinical working procedure 300, in which the method 100 according to embodiments of the present invention can be applied.

The clinical working procedure (also “workflow”) 300 begins at reference sign 302 with one or more control points. A medical indication is identified at reference sign 304. The decision is made at reference sign 306 to capture at least one image file by way of an imaging modality, for example by way of MRT and/or CT, or capture is initiated by the medical device. Scheduling is determined at reference sign 308, and an image capture protocol (also: scan protocol) is selected at reference sign 310. At reference sign 312 the at least one image file is captured according to the selected protocol. At reference sign 314 a type of processing is selected and at reference sign 316 this is applied to the at least one captured image file. At reference sign 318, a reading sequence for the image file is selected, for example if the image file comprises a stack of image slices or 3- or multi-dimensional data and/or a time sequence of images.

At reference sign 320 the at least one image file is read by trained medical personnel, in particular a radiologist, and at reference sign 322 conclusions are drawn.

Method steps may be carried out at reference sign 320-1 during the reading phase 320 and/or at reference sign 322-1 during the conclusions phase 322 (e.g. at a reading station, in particular a computer or a device 200). In particular, the trained medical personnel, in particular a specialist physician (e.g. a radiologist and/or pathologist) may initiate a guideline check, for example by clicking on an action button. The guideline check may also be denoted “online” during the reading phase 320 and/or conclusions phase 322.

A diagnostic report type and/or medical diagnosis is selected at reference sign 324 and the diagnostic report drafted over a phase 326. The diagnostic report is initialed at reference sign 328 (for example by the author and/or creator of the diagnostic report).

As soon as the medical report has been initialed, in particular by a specialist physician (e.g. at the reading workstation), an automatic guideline review (and/or a review of compliance with the guidelines) can be initiated at reference sign 328-1. Alternatively or additionally, the guideline check can be automatically triggered at reference sign 328-1 if a task (e.g. the diagnostic report) is completed and/or a diagnostic report editing program is closed.

Reference sign 330 shows a phase of central quality control for medical reports of a facility. A quality control result and/or report is output at reference sign 332.

A review 330-1 of compliance with the guidelines may proceed during the quality control phase 330. A review outside or independently of the reading and reporting working procedure 320 or 322 may also be denoted “offline”. A central decision-making machine may be part of a hospital information system (HIS) and/or a radiology information system (RIS) or the like.

If reasons for deviations from the guidelines are missing from the report, the reasons can be requested subsequently, in particular during the quality control phase 330, in order to complete the documentation of patient flow.

A review 334-1 of compliance with the guidelines may be initiated at a specific decision point in the clinical guideline, for example if a therapeutic decision (also: treatment decision) is made by the attending physician (such as surgeon, radiotherapist and/or another attending physician and/or by a team of physicians in a tumor board), for example at reference sign 334. The guideline review 334-1 may proceed automatically on the basis of a control point in the working procedure, for example at reference sign 334, and/or by trained medical personnel, in particular the attending physician. The trained medical personnel, in particular the attending physician may aggregate and evaluate a plurality of individual diagnoses and/or reports in order to arrive at a treatment decision. More than one medical report, for example all the reports relevant to the patient (e.g. radiology reports, laboratory reports, and/or pathology reports) can be reviewed for compliance with the guideline, and in particular for the presence of all relevant values. In this way, the attending physician can judge whether information might or might not possibly be missing.

A report's match with a guideline may for example be evaluated by training an (in particular AI) model. The (in particular AI) model can be trained by comparing acceptable (e.g. as meeting quality standards on the basis of quality control) reports with the corresponding requirements from the corresponding guideline and/or SOP.

An (in particular automated) guideline review can enable improved quality of the results and/or conclusions from the image file (and/or further investigations and/or diagnostic reports). Alternatively or additionally, the (in particular automated) guideline review can enable confirmation and/or documentation of guideline compliance. Furthermore alternatively or additionally, the (in particular automated) guideline review can increase transparency (e.g. of the diagnosis and/or treatment decision). Furthermore alternatively or additionally, explicable results can encourage acceptance of the (in particular automatic) reviews, for example of the guideline reviews.

It should finally be noted that the description of the present invention and the exemplary embodiments should in principle not be understood as limiting with regard to a specific physical implementation of the present invention. All the features explained and shown in connection with individual embodiments of the present invention may be provided in different combinations in the subject matter according to the present invention in order simultaneously to achieve the advantageous effects thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, Cif, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The scope of protection of the present invention, which is not limited by the features explained in the description or shown in the figures, is defined by the following claims.

In particular, it is obvious to a person skilled in the art that embodiments of the present invention are not only applicable to image files from an individual image-forming medical device, but also for a combination of medical imaging devices and a combination of other medical diagnostic techniques. Furthermore, the components of the device and/or system can be embodied in distributed manner on a plurality of physical products.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A computer-implemented method for safety checking a medical diagnostic report for a patient based on an image file acquired using a medical imaging device, the computer-implemented method comprising:
reading in sensor data relating to the image file;
assigning the sensor data to a first position of an anatomical structure of the patient;
extracting at least one second position of the anatomical structure from the medical diagnostic report;
comparing the first position and the at least one second position; and
outputting a warning signal in response to determining that the first position does not match the at least one second position.

2. The method as claimed in claim 1, wherein the sensor data includes at least one of:
a duration of display, on a screen, of an image slice within an image stack of medical images of the image file;
movement data from an input device on a display of an image or image slice of the image file;
movement data from gaze detection on the display of the image or image slice of the image file;
mark-ups of a region of interest on the display of the image or image slice of the image file;
measurements of at least one of a distance, a diameter or a density on the display of the image or image slice of the image file;
annotations about the display of the image or image slice of the image file; or
operating data with regard to display of at least one image of the image file.

3. The method as claimed in claim 1, wherein the assigning the sensor data to the first position of the anatomical structure comprises:
identifying anatomical landmarks or segmenting at least one organ of the patient.

4. The method as claimed in claim 1, wherein the assigning the sensor data to the first position of the anatomical structure comprises:
outputting a result of the assigning in a first coding scheme.

5. The method as claimed in claim 4, wherein the extracting the at least one second position of the anatomical structure from the medical diagnostic report comprises:
outputting the at least one second position in a second coding scheme.

6. The method as claimed in claim 5, wherein the comparing the first position and the at least one second position comprises:
comparing the first coding scheme and the second coding scheme.

7. The method as claimed in claim 1, wherein the outputting of the warning signal comprises at least one of:
outputting, on a display of an image or a slice of a 3D image file, the first position assigned to the sensor data for which the comparing did not identify a corresponding second position extracted from the medical diagnostic report;
text outputting a missing match with regard to the first position assigned to the sensor data; or
blocking at least one of completing or signing the medical diagnostic report.

8. The method as claimed in claim 1, wherein the medical imaging device is one of:
an X-ray machine,
a magnetic resonance tomograph,
a positron emission tomograph,
an angiography system,
a computed tomograph, or
an ultrasound scanner.

9. The method as claimed in claim 1, further comprising:
reading in further patient data, wherein the further patient data has been collected independently of the image file;
extracting at least one third position of the anatomical structure from the further patient data;
comparing the at least one third position extracted from the further patient data with at least one of the first position or the at least one second position; and
outputting an indication whether the comparing yields a match between the at least one third position and the at least one of the first position and the at least one second position, wherein the indication is directed toward reviewing a consistency of the medical diagnostic report with the further patient data.

10. The method as claimed in claim 1, further comprising:
reading in a medical guideline, which is classed as potentially relevant at least to at least one of the image file, the sensor data or the first position of the anatomical structure;
reviewing the medical diagnostic report with regard to compliance with the medical guideline; and
outputting a warning signal in response to determining that the medical diagnostic report is not compliant with the medical guideline.

11. The method as claimed in claim 10, wherein the reviewing the medical diagnostic report with regard to compliance with the medical guideline utilizes an algorithm trained by at least one of machine learning or artificial intelligence.

12. A device for safety checking a medical diagnostic report for a patient based on an image file acquired using a medical imaging device, the device comprising:
a sensor interface configured to receive, from at least one sensor, sensor data relating to the image file;
an assignment unit configured to assign the sensor data to a first position of an anatomical structure of the patient;
an extraction unit configured to extract at least one second position of the anatomical structure of the patient from the medical diagnostic report;
a comparison unit configured to compare the first position and the at least one second position; and
an output interface configured to output a warning signal in response to determining that the first position does not match the at least one second position.

13. The device as claimed in claim 12, wherein the at least one sensor includes at least one of:
at least one of a motion sensor or an input sensor of an input interface, wherein the input interface includes at least one of a computer mouse, a trackpad, a trackball, a joystick, a keyboard, or a touch-sensitive screen;
an eye tracking sensor; or
a timer configured to measure a duration of viewing of a slice from an image stack.

14. A device for safety checking a medical diagnostic report for a patient based on an image file acquired using a medical imaging device, the device configured to carry out the computer-implemented method as claimed in claim 2.

15. A system for safety checking a medical diagnostic report, the system comprising:
at least one sensor configured to acquire sensor data relating to an image file;
the device as claimed in claim 12, the sensor interface configured to read in the sensor data from the at least one sensor; and
a warning signal output device configured to output the warning signal based on the warning signal provided at the output interface of the device.

16. A non-transitory computer program product comprising commands that, when executed by a computer, cause the computer to carry out the computer-implemented method as claimed in claim 1.

17. A non-transitory computer-readable storage medium storing commands that, when executed by a computer, cause the computer to carry out the computer-implemented method of claim 1.

18. The method as claimed in claim 4, wherein the first coding scheme includes at least one of SNOMED or RadLex.

19. The method as claimed in claim 1, wherein the extracting the at least one second position of the anatomical structure from the medical diagnostic report comprises:
linguistic data processing of text of the medical diagnostic report to extract the at least one second position; and
encoding the at least one second position extracted from the linguistically data-processed text.

20. A device for safety checking a medical diagnostic report for a patient based on an image file acquired using a medical imaging device, the device comprising:
a memory storing computer-executable instructions; and
at least one processor configured to execute the computer-executable instructions to cause the device to
receive, from at least one sensor, sensor data relating to the image file,
assign the sensor data to a first position of an anatomical structure of the patient,
extract at least one second position of the anatomical structure of the patient from the medical diagnostic report,
compare the first position and the at least one second position, and
output a warning signal in response to determining that the first position does not match the at least one second position.
